SEQUENCE LISTING (22 sequences)

SEQ ID NO: 1 - protein, 900 aa, artificial sequence
  chimeric antigen receptor
NYIKGDPYFAEYATKLSFILNSSDANNPSEKIQKNNDEVCNCNESGIASVEQEQISDPSSNKTCITHSSIKANKKKVCKHVKLGVRENDKDLRVCVIEHTSLSGVENCCCQDFLRILQENCSDNKSGSSSNGSCNNKNQEACEKNLEKVLASLTNCYKCDKCKSEQSKKNNKNWIWKKSSGKEGGLQKEYANTIGLPPRTQSLCLVVCLDEKGKKTQELKNIRTNSELLKEWIIAAFHEGKNLKPSHEKKNDDNGKKLCKALEYSFADYGDLIKGTSIWDNEYTKDLELNLQKIFGKLFRKYIKKNNTAEQDTSYSSLDELRESWWNTNKKYIWLAMKHGAGMNSTTCCGDGSVTGSGSSCDDIPTIDLIPQYLRFLQEWVEHFCKQRQEKVKPVIENCKSCKESGGTCNGECKTECKNKCEVYKKFIEDCKGGDGTAGSSWVKRWDQIYKRYSKYIEDAKRNRKAGTKNCGPSSTTNAAENKCVQSDIDSFFKHLIDIGLTTPSSYLSIVLDDNICGADKAPWTTYTTYTTTEKCNKETDKSKLQQCNTAVVVNVPSPLGNTPHGYKYACQCKIPTNEETCDDRKEYMNQWSCGSARTMKRGYKNDNYELCKYNGVDVKPTTVRSNSSKLDTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 2 - DNA, 2700 bp, artificial sequence
  chimeric antigen receptor
aactacatcaagggcgacccctacttcgccgagtacgccaccaagctgagcttcatcctgaacagcagcgacgccaacaaccccagcgagaagatccagaagaacaacgacgaggtgtgcaactgcaacgagagcggcatcgccagcgtggagcaggagcagatcagcgaccccagcagcaacaagacctgcatcacccacagcagcatcaaggccaacaagaagaaggtgtgcaagcacgtgaagctgggcgtgcgcgagaacgacaaggacctgcgcgtgtgcgtgatcgagcacaccagcctgagcggcgtggagaactgctgctgccaggacttcctgcgcatcctgcaggagaactgcagcgacaacaagagcggcagcagcagcaacggcagctgcaacaacaagaaccaggaggcctgcgagaagaacctggagaaggtgctggccagcctgaccaactgctacaagtgcgacaagtgcaagagcgagcagagcaagaagaacaacaagaactggatctggaagaagagcagcggcaaggagggcggcctgcagaaggagtacgccaacaccatcggcctgcccccccgcacccagagcctgtgcctggtggtgtgcctggacgagaagggcaagaagacccaggagctgaagaacatccgcaccaacagcgagctgctgaaggagtggatcatcgccgccttccacgagggcaagaacctgaagcccagccacgagaagaagaacgacgacaacggcaagaagctgtgcaaggccctggagtacagcttcgccgactacggcgacctgatcaagggcaccagcatctgggacaacgagtacaccaaggacctggagctgaacctgcagaagatcttcggcaagctgttccgcaagtacatcaagaagaacaacaccgccgagcaggacaccagctacagcagcctggacgagctgcgcgagagctggtggaacaccaacaagaagtacatctggctggccatgaagcacggcgccggcatgaacagcaccacctgctgcggcgacggcagcgtgaccggcagcggcagcagctgcgacgacatccccaccatcgacctgatcccccagtacctgcgcttcctgcaggagtgggtggagcacttctgcaagcagcgccaggagaaggtgaagcccgtgatcgagaactgcaagagctgcaaggagagcggcggcacctgcaacggcgagtgcaagaccgagtgcaagaacaagtgcgaggtgtacaagaagttcatcgaggactgcaagggcggcgacggcaccgccggcagcagctgggtgaagcgctgggaccagatctacaagcgctacagcaagtacatcgaggacgccaagcgcaaccgcaaggccggcaccaagaactgcggccccagcagcaccaccaacgccgccgagaacaagtgcgtgcagagcgacatcgacagcttcttcaagcacctgatcgacatcggcctgaccacccccagcagctacctgagcatcgtgctggacgacaacatctgcggcgccgacaaggccccctggaccacctacaccacctacaccaccaccgagaagtgcaacaaggagaccgacaagagcaagctgcagcagtgcaacaccgccgtggtggtgaacgtgcccagccccctgggcaacaccccccacggctacaagtacgcctgccagtgcaagatccccaccaacgaggagacctgcgacgaccgcaaggagtacatgaaccagtggagctgcggcagcgcccgcaccatgaagcgcggctacaagaacgacaactacgagctgtgcaagtacaacggcgtggacgtgaagcccaccaccgtgcgcagcaacagcagcaagctggacaccaccacccccgccccccgcccccccacccccgcccccaccatcgccagccagcccctgagcctgcgccccgaggcctgccgccccgccgccggcggcgccgtgcacacccgcggcctggacttcgcctgcgacttctgggtgctggtggtggtgggcggcgtgctggcctgctacagcctgctggtgaccgtggccttcatcatcttctgggtgcgcagcaagcgcagccgcctgctgcacagcgactacatgaacatgaccccccgccgccccggccccacccgcaagcactaccagccctacgcccccccccgcgacttcgccgcctaccgcagcaagcgcggccgcaagaagctgctgtacatcttcaagcagcccttcatgcgccccgtgcagaccacccaggaggaggacggctgcagctgccgcttccccgaggaggaggagggcggctgcgagctgcgcgtgaagttcagccgcagcgccgacgcccccgcctaccagcagggccagaaccagctgtacaacgagctgaacctgggccgccgcgaggagtacgacgtgctggacaagcgccgcggccgcgaccccgagatgggcggcaagccccagcgccgcaagaacccccaggagggcctgtacaacgagctgcagaaggacaagatggccgaggcctacagcgagatcggcatgaagggcgagcgccgccgcggcaagggccacgacggcctgtaccagggcctgagcaccgccaccaaggacacctacgacgccctgcacatgcaggccctgcccccccgc

SEQ ID NO: 3 - protein, 2646 aa, artificial sequence
  VAR2CSA protein of Plasmodium falciparum strain      pf 3D7
MDKSSIANKIEAYLGAKSDDSKIDQSLKADPSEVQYYGSGGDGYYLRKNICKITVNHSDSGTNDPCDRIPPPYGDNDQWKCAIILSKVSEKPENVFVPPRRQRMCINNLEKLNVDKIRDKHAFLADVLLTARNEGERIVQNHPDTNSSNVCNALERSFADIADIIRGTDLWKGTNSNLEQNLKQMFAKIRENDKVLQDKYPKDQNYRKLREDWWNANRQKVWEVITCGARSNDLLIKRGWRTSGKSNGDNKLELCRKCGHYEEKVPTKLDYVPQFLRWLTEWIEDFYREKQNLIDDMERHREECTSEDHKSKEGTSYCSTCKDKCKKYCECVKKWKSEWENQKNKYTELYQQNKNETSQKNTSRYDDYVKDFFKKLEANYSSLENYIKGDPYFAEYATKLSFILNSSDANNPSEKIQKNNDEVCNCNESGIASVEQEQISDPSSNKTCITHSSIKANKKKVCKHVKLGVRENDKDLRVCVIEHTSLSGVENCCCQDFLRILQENCSDNKSGSSSNGSCNNKNQEACEKNLEKVLASLTNCYKCDKCKSEQSKKNNKNWIWKKSSGKEGGLQKEYANTIGLPPRTQSLCLVVCLDEKGKKTQELKNIRTNSELLKEWIIAAFHEGKNLKPSHEKKNDDNGKKLCKALEYSFADYGDLIKGTSIWDNEYTKDLELNLQKIFGKLFRKYIKKNNTAEQDTSYSSLDELRESWWNTNKKYIWLAMKHGAGMNSTTCCGDGSVTGSGSSCDDIPTIDLIPQYLRFLQEWVEHFCKQRQEKVKPVIENCKSCKESGGTCNGECKTECKNKCEVYKKFIEDCKGGDGTAGSSWVKRWDQIYKRYSKYIEDAKRNRKAGTKNCGPSSTTNAAENKCVQSDIDSFFKHLIDIGLTTPSSYLSIVLDDNICGADKAPWTTYTTYTTTEKCNKETDKSKLQQCNTAVVVNVPSPLGNTPHGYKYACQCKIPTNEETCDDRKEYMNQWSCGSARTMKRGYKNDNYELCKYNGVDVKPTTVRSNSSKLDDKDVTFFNLFEQWNKEIQYQIEQYMTNTKISCNNEKNVLSRVSDEAAQPKFSDNERDRNSITHEDKNCKEKCKCYSLWIEKINDQWDKQKDNYNKFQRKQIYDANKGSQNKKVVSLSNFLFFSCWEEYIQKYFNGDWSKIKNIGSDTFEFLIKKCGNDSGDGETIFSEKLNNAEKKCKENESTNNKMKSSETSCDCSEPIYIRGCQPKIYDGKIFPGKGGEKQWICKDTIIHGDTNGACIPPRTQNLCVGELWDKRYGGRSNIKNDTKESLKQKIKNAIQKETELLYEYHDKGTAIISRNPMKGQKEKEEKNNDSNGLPKGFCHAVQRSFIDYKNMILGTSVNIYEYIGKLQEDIKKIIEKGTTKQNGKTVGSGAENVNAWWKGIEGEMWDAVRCAITKINKKQKKNGTFSIDECGIFPPTGNDEDQSVSWFKEWSEQFCIERLQYEKNIRDACTNNGQGDKIQGDCKRKCEEYKKYISEKKQEWDKQKTKYENKYVGKSASDLLKENYPECISANFDFIFNDNIEYKTYYPYGDYSSICSCEQVKYYEYNNAEKKNNKSLCHEKGNDRTWSKKYIKKLENGRTLEGVYVPPRRQQLCLYELFPIIIKNKNDITNAKKELLETLQIVAEREAYYLWKQYHAHNDTTYLAHKKACCAIRGSFYDLEDIIKGNDLVHDEYTKYIDSKLNEIFDSSNKNDIETKRARTDWWENEAIAVPNITGANKSDPKTIRQLVWDAMQSGVRKAIDEEKEKKKPNENFPPCMGVQHIGIAKPQFIRWLEEWTNEFCEKYTKYFEDMKSNCNLRKGADDCDDNSNIECKKACANYTNWLNPKRIEWNGMSNYYNKIYRKSNKESEDGKDYSMIMEPTVIDYLNKRCNGEINGNYICCSCKNIGENSTSGTVNKKLQKKETQCEDNKGPLDLMNKVLNKMDPKYSEHKMKCTEVYLEHVEEQLKEIDNAIKDYKLYPLDRCFDDKSKMKVCDLIGDAIGCKHKTKLDELDEWNDVDMRDPYNKYKGVLIPPRRRQLCFSRIVRGPANLRNLKEFKEEILKGAQSEGKFLGNYYNEDKDKEKALEAMKNSFYDYEYIIKGSDMLTNIQFKDIKRKLDRLLEKETNNTEKVDDWWETNKKSIWNAMLCGYKKSGNKIIDPSWCTIPTTETPPQFLRWIKEWGTNVCIQKEEHKEYVKSKCSNVTNLGAQESESKNCTSEIKKYQEWSRKRSIQWEAISEGYKKYKGMDEFKNTFKNIKEPDANEPNANEYLKKHCSKCPCGFNDMQEITKYTNIGNEAFKQIKEQVDIPAELEDVIYRLKHHEYDKGNDYICNKYKNINVNMKKNNDDTWTDLVKNSSDINKGVLLPPRRKNLFLKIDESDICKYKRDPKLFKDFIYSSAISEVERLKKVYGEAKTKVVHAMKYSFADIGSIIKGDDMMENNSSDKIGKILGDGVGQNEKRKKWWDMNKYHIWESMLCGYKHAYGNISENDRKMLDIPNNDDEHQFLRWFQEWTENFCTKRNELYENMVTACNSAKCNTSNGSVDKKECTEACKNYSNFILIKKKEYQSLNSQYDMNYKETKAEKKESPEYFKDKCNGECSCLSEYFKDETRWKNPYETLDDTEVKNNCMCKPPPPASNSGREL

SEQ ID NO: 4 - DNA, 7938 bp, artificial sequence
  VAR2CSA protein of Plasmodium falciparum strain      pf 3D7
atggacaagtcctctatcgccaacaagatcgaagcctacctgggagccaagtctgacgactctaagatcgaccagtctctgaaggccgacccctctgaagtccagtactacggatctggtggcgacggttactacctgaggaagaacatctgcaagatcaccgtcaaccactctgactctggcaccaacgacccctgcgacaggatcccccctccctacggcgacaacgaccagtggaagtgcgccatcatcctgtctaaggtgtccgaaaagcccgaaaacgtgttcgtcccccctaggcgccagaggatgtgcatcaacaacctggaaaagctgaacgtcgacaagatcagggacaagcacgccttcctcgccgacgtcctgctgaccgctaggaacgaaggcgaaaggatcgtccagaaccaccccgacaccaactcttctaacgtctgcaacgccctggaacgctctttcgccgacatcgctgacatcatccgcggaaccgacctgtggaagggcaccaactctaacctggaacagaacctgaagcagatgttcgccaagatccgcgaaaacgacaaggtcctgcaggataagtaccccaaggaccagaactaccgcaagctgcgcgaggactggtggaacgccaacaggcagaaggtctgggaagtcatcacttgcggagccaggtctaacgacctgctgatcaagaggggatggcgcacctctggaaagtctaacggcgacaacaagctggaactgtgccgcaagtgcggacactacgaagaaaaggtccccaccaagctggactacgtcccccagttcctgcgctggctgaccgaatggatcgaggacttctacagggaaaagcagaacctgatcgacgacatggaaaggcacagggaagaatgcacctctgaggaccacaagtctaaggaaggcacctcttactgctctacctgcaaggacaagtgcaagaagtactgcgagtgcgtcaagaagtggaagtctgaatgggagaaccagaagaacaagtacaccgaactgtaccagcagaacaagaacgaaacctctcagaagaacacctctcgctacgacgactacgtcaaggacttcttcaagaagctcgaggccaactactcctccctggaaaactacatcaagggcgacccctacttcgccgaatacgctaccaagctgtccttcatcctgaactcttccgacgccaacaacccctctgagaagatccaaaagaacaacgacgaagtctgcaactgcaacgaatctggaatcgcctctgtcgaacaagagcagatctctgacccctctagcaacaagacctgcatcacccactcttctatcaaggccaacaagaagaaggtctgcaagcacgtcaagctgggagtcagagagaacgacaaggacctccgcgtctgcgtcatcgaacacacctccctgtctggtgtcgaaaactgctgctgccaggacttcctgaggatcctgcaagaaaactgctccgacaacaagtccggatcttcttctaacggctcctgcaacaacaagaaccaggaagcctgcgagaagaacctcgaaaaggtcctggcctctctgaccaactgctacaagtgcgacaagtgcaagtctgagcagagcaagaagaacaacaagaactggatctggaagaagtcctccggaaaggaaggtggactgcagaaggaatacgccaacaccatcggactgccccccaggacccagtctctgtgcctggtcgtctgcctggacgaaaagggaaagaagacccaagagctgaagaacatccgcaccaactccgaactgctgaaggaatggatcatcgccgccttccacgagggaaagaacctcaagccctctcacgaaaagaagaacgacgacaacggcaagaagctgtgcaaggccctcgagtactccttcgccgactacggcgacctgatcaagggaacctctatctgggacaacgagtacaccaaggacttggaactgaacctgcaaaagatcttcggcaagctgttccgcaagtacatcaagaagaacaacaccgccgagcaggacacctcctactctagcctggacgagctgagggaatcttggtggaacactaacaagaagtacatctggctggccatgaagcacggtgccggaatgaactctaccacttgctgcggcgacggatctgtgaccggatctggatcttcttgcgacgacatccccaccatcgacctgatccctcagtacctgcgcttcctgcaagagtgggtcgaacacttctgcaagcagaggcaagagaaggtcaagcccgtcatcgagaactgcaagtcttgcaaggaaagcggaggaacctgcaacggcgaatgcaagaccgagtgcaagaacaagtgtgaagtctacaagaagttcatcgaggactgcaagggtggcgacggaaccgccggatcttcctgggtcaagcgctgggaccagatctacaagaggtactctaagtacatcgaggacgccaagaggaaccgcaaggccggcaccaagaactgcggaccctcttctaccaccaacgccgccgaaaacaagtgcgtccagtctgacatcgactcattcttcaagcacctcatcgacatcggactgaccaccccctcctcttacctgtctatcgtcctggacgacaacatctgcggagccgacaaggccccctggaccacctacaccacttacaccaccaccgaaaagtgcaacaaggaaaccgacaagtccaagctccagcagtgcaacaccgctgtcgtcgtcaacgtcccctctcccctgggaaacaccccccacggatacaagtacgcctgccagtgcaagatccccaccaacgaagaaacctgcgacgaccgcaaggagtacatgaaccagtggtcttgcggatctgccaggaccatgaagaggggatacaagaacgacaactacgaactgtgcaagtacaacggtgtcgacgtcaagcccaccaccgtcaggtctaactcctctaagctggacgacaaggacgtcaccttcttcaacctgttcgagcagtggaacaaggaaatccagtaccagatcgagcagtacatgaccaacaccaagatcagctgcaacaacgaaaagaacgtcctgtctcgcgtcagcgacgaagccgcccagcccaagttctctgacaacgaaagggaccgcaactctatcacccacgaggacaagaactgcaaggaaaagtgcaagtgctactccctgtggatcgagaagatcaacgaccaatgggacaagcagaaggacaactacaacaagttccagcgcaagcagatctacgacgctaacaagggatcccaaaacaagaaggtcgtcagcctgtccaacttcctgttcttcagctgctgggaagagtacatccagaagtacttcaacggcgactggtccaagatcaagaacatcggatctgacaccttcgagttcctcatcaagaagtgcggaaacgactctggcgacggcgaaaccatcttctctgagaagctgaacaacgccgagaagaagtgcaaggaaaacgagtccaccaacaacaagatgaagtcctctgaaacctcctgcgactgctctgaacccatctacatcaggggatgccagcccaagatctacgacggaaagatcttccccggaaagggcggagaaaagcagtggatctgcaaggacaccatcatccacggcgacaccaacggtgcctgcatccctcctaggacccagaacctgtgcgtcggagaactgtgggacaagcgctacggtggaaggtctaacatcaagaacgacaccaaggaatccctgaagcagaagatcaagaacgccatccagaaggaaaccgaactgctgtacgagtaccacgacaagggaaccgccatcatctctaggaaccccatgaagggacagaaggaaaaggaagagaagaacaacgactccaacggactgcccaagggattctgccacgccgtccagcgcagcttcatcgactacaagaacatgatcctgggcacctccgtcaacatctacgagtacatcggaaagctgcaagaggacatcaagaagatcatcgagaagggaaccaccaagcagaacggaaagaccgtcggaagcggagccgaaaacgtcaacgcctggtggaagggaatcgaaggcgagatgtgggacgccgtccgctgcgccatcactaagatcaacaagaagcagaagaagaacggcaccttctctatcgacgagtgcggaatcttcccccccaccggaaacgacgaggaccagtctgtctcctggttcaaggaatggtccgaacagttctgcatcgaaaggctgcagtacgagaagaacatcagggacgcctgcaccaacaacggacagggcgacaagatccaaggcgactgcaagcgcaagtgcgaagagtacaagaagtacatcagcgagaagaagcaagagtgggacaagcaaaagactaagtacgagaacaagtacgtcggaaagtctgcctctgacctgctcaaggaaaactaccccgaatgcatctctgccaacttcgacttcatcttcaacgacaacatcgagtacaagacctactacccttacggcgactactcttccatctgctcttgcgaacaagtgaagtactacgagtacaacaacgctgaaaagaagaacaacaagtccctgtgccacgagaagggaaacgaccgcacctggtctaagaagtacatcaagaagctcgagaacggaaggaccctggaaggcgtctacgtcccaccccgcaggcagcagctgtgcctgtacgaactgttccccatcatcatcaagaacaagaacgacatcaccaacgccaagaaggaattgctggaaaccctgcagatcgtcgccgaaagggaagcctactacctctggaagcagtaccacgcccacaacgacaccacctacctggcccacaagaaggcctgctgcgctatcaggggatctttctacgacctcgaggatatcatcaagggcaacgacctggtccacgacgagtacaccaagtacatcgactctaagctgaacgagatcttcgactcctccaacaagaacgacatcgaaactaagagggccaggaccgactggtgggaaaacgaagctatcgccgtccccaacatcaccggtgccaacaagtctgaccccaagaccatcaggcagctcgtctgggacgccatgcagtctggtgtccgcaaggccatcgacgaagagaaggaaaagaagaagcccaacgaaaacttcccaccctgcatgggagtccagcacatcggaatcgccaagccccagttcatccgttggctggaagaatggaccaacgagttctgcgagaagtacactaagtacttcgaggacatgaagtccaactgcaacctgcgcaagggcgccgacgactgcgacgacaactctaacatcgaatgcaagaaggcttgcgccaactacaccaactggctgaaccccaagaggatcgaatggaacggaatgtccaactactacaacaagatctaccgcaagtccaacaaggaatctgaggacggaaaggactactcaatgatcatggaacccaccgtgatcgactacctgaacaagaggtgtaacggcgagatcaacggcaactacatctgctgctcttgcaagaacatcggcgagaactctacctctggcaccgtgaacaagaagctgcaaaagaaggaaactcagtgcgaggacaacaagggacccctggacctgatgaacaaggtcctcaacaagatggaccccaagtactctgagcacaagatgaagtgcaccgaggtctacctggaacacgtcgaagaacagttgaaggaaatcgacaacgccatcaaggactacaagctgtaccctctggaccgctgcttcgacgacaagagcaagatgaaggtttgcgacctcatcggcgacgccatcggatgcaagcacaagaccaagctcgacgaactggacgagtggaacgacgtcgacatgagggacccttacaacaagtacaagggcgtcctgatccccccaaggcgcaggcagctgtgcttctctaggatcgtcaggggacccgccaacctgaggaacctgaaggagttcaaggaagagatcctgaagggcgcccagtctgagggaaagttcctgggaaactactacaacgaggacaaggacaaggaaaaggccctggaagccatgaagaacagcttctacgactacgagtacatcatcaagggctctgacatgctgaccaacatccagttcaaggacatcaagcgcaagctggacaggctgctggaaaaggaaactaacaacaccgaaaaggtggacgactggtgggagactaacaagaagagtatctggaacgccatgttgtgcggttacaagaagtccggcaacaagatcatcgacccctcttggtgcaccatccctaccaccgaaaccccccctcagttcttgcgttggatcaaggaatggggcaccaacgtctgcatccaaaaggaagaacacaaggaatacgtcaagagcaagtgctccaacgtcaccaacctcggagcccaagaatctgaatctaagaactgcacctccgagatcaagaagtaccaagagtggtcccgcaagcgctctatccagtgggaagccatctctgagggctacaagaagtacaagggtatggacgagttcaagaacaccttcaagaacatcaaggaacccgacgccaacgagcccaacgccaacgaatacctgaagaagcactgctctaagtgcccctgcggattcaacgacatgcaagaaatcactaagtacaccaacatcggcaacgaggccttcaagcagatcaaggaacaggtcgacatccccgccgaattggaggacgtcatctacaggctgaagcaccacgaatacgacaagggaaacgactacatctgcaacaagtacaagaacatcaacgtcaacatgaagaagaacaacgacgacacctggaccgacctggtcaagaactcttctgacatcaacaagggtgtcctgctgccccctcgccgcaagaacctgttcctgaagatcgacgaatccgacatctgcaagtacaagagggaccctaagctgttcaaggacttcatctactcttccgccatctccgaagtcgaacgcctcaagaaggtctacggcgaggccaagaccaaggtcgtccacgctatgaagtactcattcgcagacatcggctccatcatcaagggcgacgacatgatggaaaacaacagctctgacaagatcggcaagatcctgggcgacggtgtcggccagaacgaaaagcgcaagaagtggtgggacatgaacaagtaccacatctgggagtctatgctgtgcggctacaagcacgcttacggaaacatctctgagaacgaccgaaagatgctggacatccccaacaacgacgacgaacaccagttcctccgctggttccaagaatggactgaaaacttctgcaccaagcgcaacgagctgtacgaaaacatggtcaccgcctgcaactctgccaagtgcaacacttctaacggatccgtggacaagaaggaatgcaccgaagcctgcaagaactactccaacttcatcctgatcaagaagaaggaataccagtccctgaactcccagtacgacatgaactacaaggaaactaaggccgaaaagaaggaatcccccgagtacttcaaggacaagtgtaacggcgaatgctcttgcctgagcgaatacttcaaggacgaaaccaggtggaagaacccctacgaaaccctcgacgacaccgaagtcaagaacaactgcatgtgcaagcccccaccccctgcctctaacagcggccgcgagctc

SEQ ID NO: 5 - protein, 2649 aa, artificial sequence
  VAR2CSA protein of Plasmodium falciparum strain      pf FCR3
MDSTSTIANKIEEYLGAKSDDSKIDELLKADPSEVEYYRSGGDGDYLKNNICKITVNHSDSGKYDPCEKKLPPYDDNDQWKCQQNSSDGSGKPENICVPPRRERLCTYNLENLKFDKIRDNNAFLADVLLTARNEGEKIVQNHPDTNSSNVCNALERSFADLADIIRGTDQWKGTNSNLEKNLKQMFAKIRENDKVLQDKYPKDQKYTKLREAWWNANRQKVWEVITCGARSNDLLIKRGWRTSGKSDRKKNFELCRKCGHYEKEVPTKLDYVPQFLRWLTEWIEDFYREKQNLIDDMERHREECTREDHKSKEGTSYCSTCKDKCKKYCECVKKWKTEWENQENKYKDLYEQNKNKTSQKNTSRYDDYVKDFFEKLEANYSSLENYIKGDPYFAEYATKLSFILNPSDANNPSGETANHNDEACNCNESGISSVGQAQTSGPSSNKTCITHSSIKTNKKKECKDVKLGVRENDKDLKICVIEDTSLSGVDNCCCQDLLGILQENCSDNKRGSSSNDSCDNKNQDECQKKLEKVFASLTNGYKCDKCKSGTSRSKKKWIWKKSSGNEEGLQEEYANTIGLPPRTQSLYLGNLPKLENVCEDVKDINFDTKEKFLAGCLIVSFHEGKNLKKRYPQNKNSGNKENLCKALEYSFADYGDLIKGTSIWDNEYTKDLELNLQNNFGKLFGKYIKKNNTAEQDTSYSSLDELRESWWNTNKKYIWTAMKHGAEMNITTCNADGSVTGSGSSCDDIPTIDLIPQYLRFLQEWVENFCEQRQAKVKDVITNCKSCKESGNKCKTECKTKCKDECEKYKKFIEACGTAGGGIGTAGSPWSKRWDQIYKRYSKHIEDAKRNRKAGTKNCGTSSTTNAAASTDENKCVQSDIDSFFKHLIDIGLTTPSSYLSNVLDDNICGADKAPWTTYTTYTTTEKCNKERDKSKSQSSDTLVVVNVPSPLGNTPYRYKYACQCKIPTNEETCDDRKEYMNQWSCGSARTMKRGYKNDNYELCKYNGVDVKPTTVRSNSSKLDGNDVTFFNLFEQWNKEIQYQIEQYMTNANISCIDEKEVLDSVSDEGTPKVRGGYEDGRNNNTDQGTNCKEKCKCYKLWIEKINDQWGKQKDNYNKFRSKQIYDANKGSQNKKVVSLSNFLFFSCWEEYIQKYFNGDWSKIKNIGSDTFEFLIKKCGNNSAHGEEIFSEKLKNAEKKCKENESTDTNINKSETSCDLNATNYIRGCQSKTYDGKIFPGKGGEKQWICKDTIIHGDTNGACIPPRTQNLCVGELWDKSYGGRSNIKNDTKELLKEKIKNAIHKETELLYEYHDTGTAIISKNDKKGQKGKNDPNGLPKGFCHAVQRSFIDYKNMILGTSVNIYEHIGKLQEDIKKIIEKGTPQQKDKIGGVGSSTENVNAWWKGIEREMWDAVRCAITKINKKNNNSIFNGDECGVSPPTGNDEDQSVSWFKEWGEQFCIERLRYEQNIREACTINGKNEKKCINSKSGQGDKIQGACKRKCEKYKKYISEKKQEWDKQKTKYENKYVGKSASDLLKENYPECISANFDFIFNDNIEYKTYYPYGDYSSICSCEQVKYYKYNNAEKKNNKSLCYEKDNDMTWSKKYIKKLENGRSLEGVYVPPRRQQLCLYELFPIIIKNEEGMEKAKEELLETLQIVAEREAYYLWKQYNPTGKGIDDANKKACCAIRGSFYDLEDIIKGNDLVHDEYTKYIDSKLNEIFGSSNTNDIDTKRARTDWWENETITNGTDRKTIRQLVWDAMQSGVRYAVEEKNENFPLCMGVEHIGIAKPQFIRWLEEWTNEFCEKYTKYFEDMKSKCDPPKRADTCGDNSNIECKKACANYTNWLNPKRIEWNGMSNYYNKIYRKSNKESEDGKDYSMIMAPTVIDYLNKRCHGEINGNYICCSCKNIGAYNTTSGTVNKKLQKKETECEEEKGPLDLMNEVLNKMDKKYSAHKMKCTEVYLEHVEEQLNEIDNAIKDYKLYPLDRCFDDQTKMKVCDLIADAIGCKDKTKLDELDEWNDMDLRGTYNKHKGVLIPPRRRQLCFSRIVRGPANLRSLNEFKEEILKGAQSEGKFLGNYYKEHKDKEKALEAMKNSFYDYEDIIKGTDMLTNIEFKDIKIKLDRLLEKETNNTKKAEDWWKTNKKSIWNAMLCGYKKSGNKIIDPSWCTIPTTETPPQFLRWIKEWGTNVCIQKQEHKEYVKSKCSNVTNLGAQASESNNCTSEIKKYQEWSRKRSIQWETISKRYKKYKRMDILKDVKEPDANTYLREHCSKCPCGFNDMEEMNNNEDNEKEAFKQIKEQVKIPAELEDVIYRIKHHEYDKGNDYICNKYKNIHDRMKKNNGNFVTDNFVKKSWEISNGVLIPPRRKNLFLYIDPSKICEYKKDPKLFKDFIYWSAFTEVERLKKAYGGARAKVVHAMKYSFTDIGSIIKGDDMMEKNSSDKIGKILGDTDGQNEKRKKWWDMNKYHIWESMLCGYREAEGDTETNENCRFPDIESVPQFLRWFQEWSENFCDRRQKLYDKLNSECISAECTNGSVDNSKCTHACVNYKNYILTKKTEYEIQTNKYDNEFKNKNSNDKDAPDYLKEKCNDNKCECLNKHIDDKNKTWKNPYETLEDTFKSKCDCPKPLPSPIKPDDLPPQADEPF

SEQ ID NO: 6 - DNA, 7947 bp, artificial sequence
  VAR2CSA protein of Plasmodium falciparum strain      pf FCR3
atggacagcaccagcacaatcgccaacaagatcgaggaatatctgggagctaagtccgatgacagcaagatcgacgaactgctgaaggccgatcctagcgaagtggagtactacagaagcggaggcgacggcgactacctgaagaacaacatctgcaagatcaccgtgaaccacagcgatagcggcaagtatgacccctgcgagaagaagctgcccccctacgacgacaacgaccagtggaagtgccagcagaacagcagcgacggcagcggcaagcccgagaacatctgcgtgccccccagacgggagcggctgtgcacctacaacctggaaaacctgaagttcgacaagatccgggacaacaacgccttcctggccgacgtgctgctgaccgcccggaacgagggcgagaagatcgtgcagaaccaccccgacaccaacagcagcaacgtgtgcaacgccctggaacggtccttcgctgacctggctgacatcatccggggcaccgatcagtggaagggcaccaactccaatctggaaaagaacctgaagcagatgttcgccaagatcagagaaaacgacaaggtgctgcaggacaagtaccccaaggaccagaagtacaccaagctgcgggaggcctggtggaacgccaaccggcagaaagtgtgggaagtgatcacctgtggcgccagaagcaacgatctgctgatcaagcggggctggcggaccagcggcaagagcgaccggaagaaaaacttcgagctgtgccggaagtgcggccactacgagaaagaggtgcccaccaagctggactacgtgccccagttcctgcggtggctgaccgagtggatcgaggacttctaccgggagaagcagaacctgatcgacgacatggaacggcaccgggaggaatgcaccagagaggaccacaagagcaaagagggcaccagctactgcagcacatgcaaggacaagtgcaagaaatactgcgagtgcgtgaagaaatggaaaaccgagtgggagaaccaggaaaacaagtacaaggacctgtacgagcagaacaagaacaagaccagccagaagaacaccagcagatacgacgactacgtgaaggacttcttcgagaagctggaagccaactacagcagcctggaaaactacatcaagggcgacccctatttcgctgagtacgctacaaaactgagcttcatcctgaaccccagcgacgccaacaaccccagcggcgagacagccaaccacaacgacgaggcctgcaactgcaacgagagcggcatcagcagcgtgggccaggctcagacatccggccctagcagcaacaagacctgtatcacccacagctccatcaagaccaacaagaaaaaagaatgcaaggacgtgaagctgggcgtgcgggagaacgacaaggatctgaagatctgcgtgatcgaggacaccagcctgagcggcgtggacaactgctgctgccaggatctgctgggcatcctgcaggaaaactgcagcgacaacaagcggggcagcagctccaacgacagctgcgacaataagaaccaggacgagtgccagaaaaagctggaaaaggtgttcgccagcctgaccaacggctacaagtgcgataagtgcaagagcggcacctcccggtccaagaagaagtggatctggaagaagtccagcggcaacgaggaaggcctgcaggaagagtacgccaacaccatcggcctgccccccaggacccagagcctgtacctgggcaatctgcccaaactggaaaacgtgtgcgaggatgtgaaggacatcaacttcgacaccaaagagaagtttctggccggctgcctgatcgtgtccttccacgagggcaagaatctgaagaagcgctacccccagaataagaacagcggcaacaaagaaaacctgtgcaaggctctggaatacagcttcgccgactacggcgacctgatcaagggcacctccatctgggacaacgagtacacaaaggacctggaactgaatctgcagaacaacttcggcaagctgttcggcaagtacatcaagaagaacaataccgccgagcaggacacctcctacagctccctggacgagctgcgcgagtcttggtggaataccaataagaagtacatctggaccgccatgaagcacggcgccgagatgaacatcaccacctgtaacgccgacggctccgtgaccggcagcggctccagctgcgacgacatccccaccatcgacctgatcccccagtacctgagatttctgcaggaatgggtcgagaacttctgcgagcagcggcaggccaaagtgaaggacgtgatcaccaactgcaagagctgcaaagaatccggcaacaaatgcaagaccgagtgcaaaaccaagtgcaaggatgagtgcgagaagtacaagaagttcatcgaggcctgcggcacagccggcggaggcatcggaacagccggcagcccctggtccaagagatgggaccagatctacaagcggtacagcaagcacatcgaggacgccaagcggaaccggaaggccggcaccaagaactgcggcaccagctccaccaccaacgccgctgccagcaccgacgagaataagtgcgtgcagagcgacatcgacagctttttcaagcacctgatcgatatcggcctgaccacccccagcagctacctgagcaacgtgctggacgacaacatctgtggcgccgacaaggccccctggacaacctatacaacatacactacaaccgagaagtgcaacaaagagcgggacaagagcaagagccagagcagcgacaccctggtggtggtgaacgtgcccagccccctgggcaacacaccctaccggtacaagtacgcctgccagtgcaagatccccaccaacgaggaaacatgcgacgaccggaaagaatacatgaaccagtggtcctgcgggagcgctcggaccatgaagagagggtataagaacgataactacgaactgtgcaagtacaacggcgtggatgtgaagcccaccaccgtgcggagcaactccagcaagctggacggcaacgacgtgaccttcttcaacctgttcgagcagtggaacaaagaaatccagtaccagatcgagcagtacatgaccaacgccaacatcagctgcatcgatgagaaagaagtgctggacagcgtctccgacgagggcacccccaaagtgcggggaggctacgaggacggccggaacaacaacacagatcagggcacaaattgcaaagaaaagtgcaagtgctacaagctgtggatcgagaagatcaacgatcagtggggcaagcagaaggacaactacaacaagttccggtccaagcagatctacgacgccaataagggcagccagaacaaaaaggtggtgtccctgagcaacttcctgttcttcagctgctgggaggaatacatccagaagtacttcaacggcgattggagcaagatcaagaacatcggctccgacaccttcgagtttctgatcaagaagtgcggcaacaacagcgcccacggcgaggaaatcttcagcgagaagctgaagaacgccgagaagaagtgcaaagagaacgagagcaccgacaccaatatcaacaagagcgagacatcctgcgacctgaacgccaccaactacatccggggctgccagagcaagacctacgacggcaagatcttccccggcaagggcggcgagaagcagtggatttgcaaggacaccatcatccacggcgacaccaacggcgcctgcatcccccctcgcacccagaacctgtgcgtgggcgagctgtgggacaagtcctacggcggcagatccaacatcaagaacgataccaaagaactgctgaaagagaagattaagaacgccatccacaaagagacagagctgctgtacgagtaccacgacaccggcaccgccatcatcagcaagaatgacaagaagggccagaagggcaagaacgaccctaacggcctgcccaaaggcttctgtcatgctgtgcagcggagcttcatcgactacaagaacatgatcctgggcaccagcgtgaacatctacgagcacatcggcaagctgcaggaagatatcaagaagatcatcgaaaagggcacccctcagcagaaggataagatcggcggcgtgggcagcagcaccgagaacgtgaacgcttggtggaagggaatcgagcgggagatgtgggacgccgtgagatgcgccatcaccaagatcaataagaagaataacaacagcatcttcaatggcgacgagtgcggcgtgagcccccccaccggcaacgatgaggaccagagcgtgtcctggttcaaagagtggggcgagcagttctgcatcgagcggctgagatacgaacagaacatcagagaggcctgcaccatcaacggcaaaaacgagaagaaatgcatcaacagcaagtccggccagggcgataagatccagggcgcctgcaagcggaagtgtgaaaagtataagaagtatatcagcgagaaaaaacaggaatgggacaagcagaaaaccaagtacgagaacaaatacgtgggcaagagcgccagcgatctgctgaaagaaaactaccccgagtgcatcagcgccaacttcgacttcatcttcaacgacaatatcgagtacaagacctactacccctacggcgattacagcagcatctgcagctgcgaacaggtgaagtactataagtacaacaatgccgagaaaaagaacaacaagagcctgtgctacgagaaggacaatgacatgacctggtctaagaaatatatcaagaagctggaaaacggccggtccctggaaggcgtgtacgtgccccctcggcggcagcagctgtgtctgtacgagctgttccctatcatcatcaaaaacgaagagggcatggaaaaggccaaagaggaactgctggaaaccctgcagatcgtggccgagcgcgaggcctactacctgtggaagcagtacaaccccacaggcaaagggattgatgatgccaacaagaaggcctgctgcgccatcagaggcagcttctacgacctggaagatattatcaagggcaacgacctggtgcacgacgagtacaccaaatacatcgactccaagctgaacgagatcttcggctccagcaacaccaacgacatcgataccaagcgggccagaaccgattggtgggagaacgagacaatcaccaacggcaccgacagaaagaccatccggcagctggtgtgggatgccatgcagagcggagtgcgatatgctgtggaggaaaagaacgagaacttccccctgtgcatgggcgtggagcacattggcattgccaagccccagttcatcagatggctggaagagtggaccaacgagttctgcgagaaatacaccaagtacttcgaggacatgaagtccaagtgcgacccccccaagagggccgacacatgcggcgacaactccaacatcgagtgcaagaaggcttgcgccaactacaccaactggctgaaccccaagcggatcgagtggaacggcatgagcaactactacaataagatctaccggaagtctaacaaagagagcgaggacggcaaggactacagcatgatcatggcccccaccgtgatcgattacctgaacaagcggtgccacggcgagatcaatggcaactacatctgctgcagctgcaagaatatcggcgcctacaacaccacctccggcaccgtgaacaagaagctgcagaaaaaagaaaccgagtgcgaggaagagaagggccctctggacctgatgaacgaggtgctgaacaagatggacaagaagtactccgcccacaagatgaagtgcaccgaggtgtacctggaacacgtggaggaacagctgaatgagatcgacaacgccatcaaggattacaagctgtaccccctggacagatgcttcgacgaccagaccaagatgaaagtgtgcgatctgatcgccgacgccatcggctgcaaggataagacaaagctggatgagctggacgaatggaacgacatggacctgagaggcacctacaataagcacaagggcgtgctgatcccccccaggcggagacagctgtgcttcagccggatcgtgcggggacccgccaacctgagaagcctgaacgagttcaaagaggaaatcctgaagggcgctcagtccgagggcaagttcctgggcaattactacaaagagcacaaggacaaagagaaggccctggaagccatgaagaacagcttttacgactacgaggacatcatcaagggaaccgacatgctgaccaacatcgaattcaaggatatcaagatcaagctggaccggctgctggaaaaagagacaaacaacaccaagaaagccgaggactggtggaaaaccaacaaaaagtctatttggaacgccatgctgtgcggctacaaaaagagcggcaataagatcattgaccccagctggtgcaccatccctaccaccgagacacccccccagtttctgagatggatcaaagaatggggcaccaatgtgtgcatccagaagcaggaacacaaagaatacgtcaagtctaagtgctccaacgtgaccaatctgggagcacaggccagcgagagcaacaactgcaccagcgagatcaagaaataccaggaatggtcccggaagcggagcatccagtgggagacaatcagcaagcgctacaagaaatacaagcggatggacatcctgaaggatgtgaaagagcctgacgccaatacctacctgagagaacactgcagcaagtgcccctgcggcttcaacgatatggaagagatgaacaacaacgaggacaatgagaaagaggccttcaagcagatcaaagaacaggtgaaaatccccgccgagctggaagatgtgatctacagaatcaagcaccacgagtacgacaagggcaatgactacatctgtaacaaatacaagaatatccacgaccggatgaagaaaaacaacggcaacttcgtgaccgacaatttcgtgaagaagtcctgggagatttccaatggagtgctgatccctcctcggagaaagaacctgttcctgtacatcgaccccagcaagatctgcgaatacaagaaggaccccaagctgttcaaggacttcatctattggagcgccttcaccgaggtggagcggctgaagaaggcctacggcggagccagagccaaggtggtgcacgccatgaagtatagcttcaccgacatcggcagcatcattaagggcgacgacatgatggaaaagaatagcagcgacaagatcggcaagatcctgggggacaccgacggccagaacgagaagcgcaaaaagtggtgggacatgaacaagtaccacatctgggagagcatgctgtgtggataccgggaggccgagggagatacagagactaacgaaaactgccggttccccgacatcgagagcgtgcctcagtttctgcgctggtttcaggaatggtcagagaatttttgcgatagaaggcagaagctgtacgacaagctgaacagcgagtgcatctccgccgagtgcaccaatggctccgtggacaatagcaagtgcacccacgcctgcgtgaactacaagaattacatcctgaccaaaaagaccgagtacgagatccagaccaataagtacgacaacgagtttaagaacaagaatagcaacgataaggacgcccccgactatctgaaagagaaatgcaacgacaacaagtgcgagtgcctgaataagcacattgacgacaagaacaaaacctggaagaacccctacgagacactggaagataccttcaagagcaagtgcgactgccctaagcccctgccctcccccatcaagcccgacgatctgccaccccaggccgacgagcccttc

SEQ ID NO: 7 - protein, 632 aa, artificial sequence
  peptide fragment ID1-ID2a
NYIKGDPYFAEYATKLSFILNSSDANNPSEKIQKNNDEVCNCNESGIASVEQEQISDPSSNKTCITHSSIKANKKKVCKHVKLGVRENDKDLRVCVIEHTSLSGVENCCCQDFLRILQENCSDNKSGSSSNGSCNNKNQEACEKNLEKVLASLTNCYKCDKCKSEQSKKNNKNWIWKKSSGKEGGLQKEYANTIGLPPRTQSLCLVVCLDEKGKKTQELKNIRTNSELLKEWIIAAFHEGKNLKPSHEKKNDDNGKKLCKALEYSFADYGDLIKGTSIWDNEYTKDLELNLQKIFGKLFRKYIKKNNTAEQDTSYSSLDELRESWWNTNKKYIWLAMKHGAGMNSTTCCGDGSVTGSGSSCDDIPTIDLIPQYLRFLQEWVEHFCKQRQEKVKPVIENCKSCKESGGTCNGECKTECKNKCEVYKKFIEDCKGGDGTAGSSWVKRWDQIYKRYSKYIEDAKRNRKAGTKNCGPSSTTNAAENKCVQSDIDSFFKHLIDIGLTTPSSYLSIVLDDNICGADKAPWTTYTTYTTTEKCNKETDKSKLQQCNTAVVVNVPSPLGNTPHGYKYACQCKIPTNEETCDDRKEYMNQWSCGSARTMKRGYKNDNYELCKYNGVDVKPTTVRSNSSKLD

SEQ ID NO: 8 - DNA, 1896 bp, artificial sequence
  peptide fragment ID1-ID2a
aactacatcaagggcgacccctacttcgccgagtacgccaccaagctgagcttcatcctgaacagcagcgacgccaacaaccccagcgagaagatccagaagaacaacgacgaggtgtgcaactgcaacgagagcggcatcgccagcgtggagcaggagcagatcagcgaccccagcagcaacaagacctgcatcacccacagcagcatcaaggccaacaagaagaaggtgtgcaagcacgtgaagctgggcgtgcgcgagaacgacaaggacctgcgcgtgtgcgtgatcgagcacaccagcctgagcggcgtggagaactgctgctgccaggacttcctgcgcatcctgcaggagaactgcagcgacaacaagagcggcagcagcagcaacggcagctgcaacaacaagaaccaggaggcctgcgagaagaacctggagaaggtgctggccagcctgaccaactgctacaagtgcgacaagtgcaagagcgagcagagcaagaagaacaacaagaactggatctggaagaagagcagcggcaaggagggcggcctgcagaaggagtacgccaacaccatcggcctgcccccccgcacccagagcctgtgcctggtggtgtgcctggacgagaagggcaagaagacccaggagctgaagaacatccgcaccaacagcgagctgctgaaggagtggatcatcgccgccttccacgagggcaagaacctgaagcccagccacgagaagaagaacgacgacaacggcaagaagctgtgcaaggccctggagtacagcttcgccgactacggcgacctgatcaagggcaccagcatctgggacaacgagtacaccaaggacctggagctgaacctgcagaagatcttcggcaagctgttccgcaagtacatcaagaagaacaacaccgccgagcaggacaccagctacagcagcctggacgagctgcgcgagagctggtggaacaccaacaagaagtacatctggctggccatgaagcacggcgccggcatgaacagcaccacctgctgcggcgacggcagcgtgaccggcagcggcagcagctgcgacgacatccccaccatcgacctgatcccccagtacctgcgcttcctgcaggagtgggtggagcacttctgcaagcagcgccaggagaaggtgaagcccgtgatcgagaactgcaagagctgcaaggagagcggcggcacctgcaacggcgagtgcaagaccgagtgcaagaacaagtgcgaggtgtacaagaagttcatcgaggactgcaagggcggcgacggcaccgccggcagcagctgggtgaagcgctgggaccagatctacaagcgctacagcaagtacatcgaggacgccaagcgcaaccgcaaggccggcaccaagaactgcggccccagcagcaccaccaacgccgccgagaacaagtgcgtgcagagcgacatcgacagcttcttcaagcacctgatcgacatcggcctgaccacccccagcagctacctgagcatcgtgctggacgacaacatctgcggcgccgacaaggccccctggaccacctacaccacctacaccaccaccgagaagtgcaacaaggagaccgacaagagcaagctgcagcagtgcaacaccgccgtggtggtgaacgtgcccagccccctgggcaacaccccccacggctacaagtacgcctgccagtgcaagatccccaccaacgaggagacctgcgacgaccgcaaggagtacatgaaccagtggagctgcggcagcgcccgcaccatgaagcgcggctacaagaacgacaactacgagctgtgcaagtacaacggcgtggacgtgaagcccaccaccgtgcgcagcaacagcagcaagctggac

SEQ ID NO: 9 - protein, 45 aa, artificial sequence
  hinge region of human CD8alpha
TTTPAPRPPTPAPTIASQPLSLRPEACRPAAGGAVHTRGLDFACD

SEQ ID NO: 10 - DNA, 135 bp, artificial sequence
  hinge region of human CD8alpha
accaccacccccgccccccgcccccccacccccgcccccaccatcgccagccagcccctgagcctgcgccccgaggcctgccgccccgccgccggcggcgccgtgcacacccgcggcctggacttcgcctgcgac

SEQ ID NO: 11 - protein, 27 aa, artificial sequence
  transmembrane region of human CD28
FWVLVVVGGVLACYSLLVTVAFIIFWV

SEQ ID NO: 12 - DNA, 81 bp, artificial sequence
  transmembrane region of human CD28
ttctgggtgctggtggtggtgggcggcgtgctggcctgctacagcctgctggtgaccgtggccttcatcatcttctgggtg

SEQ ID NO: 13 - protein, 113 aa, artificial sequence
  intracellular region of human CD3zeta
RVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 14 - DNA, 339 bp, artificial sequence
  intracellular region of human CD3zeta
cgcgtgaagttcagccgcagcgccgacgcccccgcctaccagcagggccagaaccagctgtacaacgagctgaacctgggccgccgcgaggagtacgacgtgctggacaagcgccgcggccgcgaccccgagatgggcggcaagccccagcgccgcaagaacccccaggagggcctgtacaacgagctgcagaaggacaagatggccgaggcctacagcgagatcggcatgaagggcgagcgccgccgcggcaagggccacgacggcctgtaccagggcctgagcaccgccaccaaggacacctacgacgccctgcacatgcaggccctgcccccccgc

SEQ ID NO: 15 - protein, 154 aa, artificial sequence
  tandem arrangement of the intracellular region      of human CD28 and human CD3zeta
RSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 16 - DNA, 462 bp, artificial sequence
  tandem arrangement of the intracellular region      of human CD28 and human CD3zeta
cgcagcaagcgcagccgcctgctgcacagcgactacatgaacatgaccccccgccgccccggccccacccgcaagcactaccagccctacgcccccccccgcgacttcgccgcctaccgcagccgcgtgaagttcagccgcagcgccgacgcccccgcctaccagcagggccagaaccagctgtacaacgagctgaacctgggccgccgcgaggagtacgacgtgctggacaagcgccgcggccgcgaccccgagatgggcggcaagccccagcgccgcaagaacccccaggagggcctgtacaacgagctgcagaaggacaagatggccgaggcctacagcgagatcggcatgaagggcgagcgccgccgcggcaagggccacgacggcctgtaccagggcctgagcaccgccaccaaggacacctacgacgccctgcacatgcaggccctgcccccccgc

SEQ ID NO: 17 - protein, 155 aa, artificial sequence
  tandem arrangement of the intracellular region      of human 4-1BB and human CD3zeta
KRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 18 - DNA, 465 bp, artificial sequence
  tandem arrangement of the intracellular region      of human 4-1BB and human CD3zeta
aagcgcggccgcaagaagctgctgtacatcttcaagcagcccttcatgcgccccgtgcagaccacccaggaggaggacggctgcagctgccgcttccccgaggaggaggagggcggctgcgagctgcgcgtgaagttcagccgcagcgccgacgcccccgcctaccagcagggccagaaccagctgtacaacgagctgaacctgggccgccgcgaggagtacgacgtgctggacaagcgccgcggccgcgaccccgagatgggcggcaagccccagcgccgcaagaacccccaggagggcctgtacaacgagctgcagaaggacaagatggccgaggcctacagcgagatcggcatgaagggcgagcgccgccgcggcaagggccacgacggcctgtaccagggcctgagcaccgccaccaaggacacctacgacgccctgcacatgcaggccctgcccccccgc

SEQ ID NO: 19 - protein, 196 aa, artificial sequence
  tandem arrangement of the intracellular region      of human CD28
RSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 20 - DNA, 588 bp, artificial sequence
  tandem arrangement of the intracellular region      of human CD28
cgcagcaagcgcagccgcctgctgcacagcgactacatgaacatgaccccccgccgccccggccccacccgcaagcactaccagccctacgcccccccccgcgacttcgccgcctaccgcagcaagcgcggccgcaagaagctgctgtacatcttcaagcagcccttcatgcgccccgtgcagaccacccaggaggaggacggctgcagctgccgcttccccgaggaggaggagggcggctgcgagctgcgcgtgaagttcagccgcagcgccgacgcccccgcctaccagcagggccagaaccagctgtacaacgagctgaacctgggccgccgcgaggagtacgacgtgctggacaagcgccgcggccgcgaccccgagatgggcggcaagccccagcgccgcaagaacccccaggagggcctgtacaacgagctgcagaaggacaagatggccgaggcctacagcgagatcggcatgaagggcgagcgccgccgcggcaagggccacgacggcctgtaccagggcctgagcaccgccaccaaggacacctacgacgccctgcacatgcaggccctgcccccccgc

SEQ ID NO: 21 - protein, 21 aa, artificial sequence
  human CD8alpha signal peptide
MALPVTALLLPLALLLHAARP

SEQ ID NO: 22 - DNA, 63 bp, Artifical Sequence
  human CD8alpha signal peptide
atggccttaccagtgaccgccttgctcctgccgctggccttgctgctccacgccgccaggccg

(19) United States
(12) Patent Application Publication
(10) Pub. No.: US 2019/0153049 A1
(43) Pub. Date: May 23, 2019

(54) ANTI-PLACENTA-CHONDROITIN-SULFATE CHIMERIC ANTIGEN RECEPTOR AND APPLICATION THEREOF

(71) Applicant: GUANGZHOU CAS LAMVAC BIOTECH CO., LTD., Guangzhou (CN)

(72) Inventors: Yongchao Yao, Guangzhou (CN); Hemei Qi, Guangzhou (CN); Wenzhong Guo, Guangzhou (CN); Li Qin, Guangzhou (CN); Xiaoping Chen, Guangzhou (CN)

(73) Assignee: GUANGZHOU CAS LAMVAC BIOTECH CO., LTD., Guangzhou (CN)

(21) Appl. No.: 15/779,382

(22) PCT Filed: Oct. 26, 2016
(86) PCT No.: PCT/CN2016/103457

§ 371 Date: May 25, 2018

(30) Foreign Application Priority Data
Nov. 26, 2015 (CN) 201510843720.9
Jan. 18, 2016 (CN) 201610032290.7

(51) Int. Cl. C07K 14/445; C07K 14/705; C07K 14/725; C12N 5/0783; A61P 35/00

(52) U.S. Cl. CPC C07K 14/445; C07K 14/70517; C07K 14/70521; C07K 2319/03; C07K 14/70532; C12N 5/0636; A61P 35/00; C07K 14/7051

(57) ABSTRACT

The present invention provides an anti-placenta chondroitin sulfate (pl-CS) chimeric antigen receptor and application thereof. The chimeric antigen receptor mainly comprises anti-pl-CS antigen recognition region, a hinge region, a transmembrane region and intracellular region, wherein the anti-pl-CS antigen recognition region is any one of a plasmodium protein VAR2CSA, a part of peptide segment of plasmodium protein VAR2CSA or a pl-CS antibody; and the part of peptide segment of the plasmodium protein VAR2CSA is a peptide segment with the number of amino acid greater than 500 in the plasmodium protein VAR2CSA.

### TECHNICAL FIELD

The present invention relates to the field of cellular immunotherapy for tumors, in particular to a chimeric antigen receptor of anti-placental-like chondroitin sulfate and application thereof, specifically to a construction method of chimeric antigen receptor T cell (CAR-T) technology based on a tumor-specific target pl-CS and its application in anti-tumor therapy.

### BACKGROUND

The chimeric antigen receptor T cell (CAR-T) technology is a novel cell therapy that is currently undergoing large-scale clinical trials. It has significant efficiency on the treatment of acute leukemia and non-Hodgkin's lymphoma, and is considered as one of the most promising methods for treating tumors. The basic technique thereof is to transform T cells into those that recognize tumor antigens in a HLA-independent manner through genetic engineering methods, allowing CAR-T cells to have stronger ability to recognize and kill tumor cells than natural T cells. The core component of CAR-T is the CAR, which typically includes: a tumor-associated antigen binding region typically derived from scFv fragment of a monoclonal antibody against the tumor-associated antigen; an extracellular hinge region; a transmembrane region and an intracellular stimulation signal region.

The key to the success of CAR-T technology is the selection of tumor antigens, i.e. the targets. The ideal antigen is an antigen that is highly expressed on the surface of tumor cells and is not expressed in normal cells. Only by selection of such antigens, CAR-T cells will be enabled to specifically bind to and kill tumor cells, rather than bind to normal cells, and thereby will not impair normal tissues. However, there are few such ideal antigens. Most antigens are highly expressed or over-expressed in tumor tissues and low expressed in normal tissues. At present, the treatment efficiency of CAR-T for solid tumors is far less than that for leukemia, one of the reasons for which is that no ideal targets have been found in solid tumors.

Another reason why CAR-T technology is not effective for treating solid tumors is the existence of a tumor immunosuppressive microenvironment. The tumor immunosuppressive microenvironment is an internal environment formed during the development and progression of a tumor and is composed of tumor cells, mesenchymal cells, immunoregulatory cells, microvessels, interstitial fluid, cytokines, and extracellular matrix, etc. Due to the existence of immune microenvironment, immune killer cells cannot reach the tumor cell sites effectively or cannot effectively kill tumor cells after reaching the target sites. To effectively treat solid tumors, CAR-T cells must break through the limits of tumor immune microenvironment and enter solid tumors to kill tumor cells.

CN 104788573 A discloses a chimeric antigen receptor hCD19scFv-CD8α-CD28-CD3 [symbol] and its use. The chimeric antigen receptor is composed of the variable regions of light and heavy chains of anti-human CD19 monoclonal antibody (hCD19scFv), the hinge region of human CD8α, the transmembrane region, and the intracellular region of human CD28 and the intracellular region of human CD3ζ in tandem arrangement. The chimeric antigen receptor is used to modify T lymphocytes, and the modified T cells (CAR-T cells) can be used to treat tumors with CD19 positive on the surface. The chimeric antigen receptor of this patent is only directed to tumors with CD19 positive on the surface, and the recognition effect is not well. Therefore, both screening suitable tumor targets and breaking through the tumor microenvironment are the keys to treating solid tumors by CAR-T technology.

Chondroitin sulfate (CS) is a kind of glycosaminoglycan that is covalently attached to proteins to form proteoglycans. Chondroitin sulfate is widely distributed on the extracellular matrix and cell surface of animal tissues, and the sugar chain is composed of disaccharide units of alternating glucuronic acid and N-acetylgalactosamine (also known as N-acetylgalactose), attached to the serine residue of the core protein through a sugar-like linking region. Chondroitin sulfate exists in all organisms other than plants, from nematodes to human beings, and plays an important role in many physiological functions. Although the backbone structure of the polysaccharide is not complicated, it shows a high degree of heterogeneity in terms of the degree of sulfation, the distribution of sulfate groups and the two epimeric uronic acids in the chain. The fine structure of chondroitin sulfate determines the specificity of the function and the interaction with various protein molecules.

There exists a special chondroitin sulfate in the placenta, the glycosylation pattern of which is different from that of conventional chondroitin sulfate. In Plasmodium falciparum, there exists a protein that is called VAR2CSA and can specifically bind to the chondroitin sulfate in placenta. On the surface of tumor cells, there exists a placental-like chondroitin sulfate (pl-CS), and therefore, pl-CS is an ideal target for the treatment of tumors with CAR-T technology.

### SUMMARY OF THE INVENTION

The present invention provides a chimeric antigen receptor of anti-placental-like chondroitin sulfate and its application in view of the facts that the selection of targets for the treatment of tumors with CAR-T technology is not ideal currently and that the therapeutic effect of CAR-T technology is impaired by tumor microenvironment. The chimeric antigen receptor of the present invention can recognize tumor cells more specifically and have better targetability to tumor microenvironment.

To achieve the purpose, the present invention adopts the following technical solutions:

In one aspect, the present invention provides a chimeric antigen receptor of anti-placental-like chondroitin sulfate, mainly comprising an antigen recognition region of anti-pl-CS, a hinge region, a transmembrane region, and an intracellular region in tandem arrangement;

Wherein, the antigen recognition region of anti-pl-CS is any one of Plasmodium falciparum protein VAR2CSA, partial peptide fragment of the Plasmodium falciparum protein VAR2CSA or pl-CS antibody;

The partial peptide fragment of the Plasmodium falciparum protein VAR2CSA is a peptide fragment having a number of amino acids greater than 500 from the Plasmodium falciparum protein VAR2CSA.

In the present invention, the partial peptide fragment of the Plasmodium falciparum protein VAR2CSA refers to a peptide fragment having a number of amino acids greater than 500 from the Plasmodium falciparum protein VAR2CSA, and may be any peptide having a number of amino acids of 500 or more than 500 from the Plasmodium falciparum protein VAR2CSA, all of which are available. In the present invention, the Plasmodium falciparum protein VAR2CSA, the partial peptide fragment ID1-ID2a of the Plasmodium falciparum protein VAR2CSA and the pl-CS antibody can specifically bind to pl-CS. pl-CS is present on almost all types of tumor cells, while not on the surface of normal cells. By selecting pl-CS as the specific target for tumors, using VAR2CSA as the recognition domain of the CAR to recognize pl-CS on tumors, that is, recognizing pl-CS by VAR2CSA, CAR-T cells can be enabled to recognize tumor cells specifically, target the tumor microenvironment more easily and enter into the tumor tissues without damaging normal cells and tissues. In addition, pl-CS is a component of the extracellular matrix of tumors and acts as the material basis for forming tumor microenvironment. Therefore, selecting pl-CS as a target is helpful for CAR-T cells to break through the tumor immunosuppressive microenvironment.

Preferably, the Plasmodium falciparum protein VAR2CSA is a VAR2CSA protein of Plasmodium falciparum pf 3D7 and/or a VAR2CSA protein of Plasmodium falciparum pf FCR3.

Preferably, the VAR2CSA protein of Plasmodium falciparum pf 3D7 has an amino acid sequence of SEQ ID NO.3, and a nucleic acid sequence of SEQ ID NO.4.

Preferably, the VAR2CSA protein of Plasmodium falciparum pf FCR3 has an amino acid sequence of SEQ ID NO.5, and a nucleic acid sequence of SEQ ID NO.6.

Preferably, the partial peptide fragment of the Plasmodium falciparum protein VAR2CSA is ID1-ID2a, which has an amino acid sequence of SEQ ID NO.7, and a nucleic acid sequence of SEQ ID NO.8.

In the present invention, the ID1-ID2a is a peptide with a smaller length and the best effect, among these partial peptide fragments.

Preferably, the hinge region is the hinge region of human CD8α.

Preferably, the hinge region of human CD8α has an amino acid sequence of SEQ ID NO.9, and a nucleic acid sequence of SEQ ID NO.10.

Preferably, the transmembrane region is the transmembrane region of human CD28.

Preferably, the transmembrane region of human CD28 has an amino acid sequence of SEQ ID NO.11, and a nucleic acid sequence of SEQ ID NO.12.

As a preferred technical solution, the intracellular region is any one of the intracellular region of human CD3ζ, the intracellular region of human CD28 or the intracellular region of human 4-1BB, or a combination of at least two thereof, for example, the intracellular region of CD3ζ, the intracellular region of human CD28, the intracellular region of human 4-1BB, the tandem arrangement of intracellular region of human CD28 and intracellular region of human CD3ζ, the tandem arrangement of intracellular region of human 4-1BB and intracellular region of human CD3ζ the tandem arrangement of intracellular region of human CD3ζ and intracellular region of human 4-1BB, or the tandem arrangement of intracellular region of human CD28, intracellular region of human 4-1BB and intracellular region of human CD3ζ, preferably the intracellular region of CD3ζ, the tandem arrangement of intracellular region of human CD28 and intracellular region of human CD3ζ, the tandem arrangement of intracellular region of human 4-1BB and intracellular region of human CD3ζ, or the tandem arrangement of intracellular region of human CD28, intracellular region of human 4-1BB and intracellular region of human CD3ζ.

Preferably, the intracellular region of human CD3ζ has an amino acid sequence of SEQ ID NO.13, and a nucleic acid sequence of SEQ ID NO.14.

Preferably, the tandem arrangement of intracellular region of human CD28 and intracellular region of human CD3ζ has an amino acid sequence of SEQ ID NO.15, and a nucleic acid sequence of SEQ ID NO.16.

Preferably, the tandem arrangement of intracellular region of human 4-1BB and intracellular region of human CD3ζ has an amino acid sequence of SEQ ID NO.17, and a nucleic acid sequence of SEQ ID NO.18.

Preferably, the tandem arrangement of intracellular region of human CD28, intracellular region of human 4-1BB and intracellular region of human CD3ζ has an amino acid sequence of SEQ ID NO.19, and a nucleic acid sequence of SEQ ID NO.20.

Preferably, the chimeric antigen receptor contains a human CD8α signal peptide at its amino terminus.

Preferably, the human CD8α signal peptide has an amino acid sequence of SEQ ID NO.21 and a nucleic acid sequence of SEQ ID NO.22.

As a preferred technical solution, the chimeric antigen receptor is composed of human CD8α signal peptide, VAR2CSA, hinge region of human CD8α, transmembrane region and intracellular region of human CD28, intracellular region of human 4-1BB and intracellular region of human CD3ζ in tandem arrangement.

Preferably, the chimeric antigen receptor has an amino acid sequence of SEQ ID NO.1 and a nucleic acid sequence of SEQ ID NO.2.

Preferably, the chimeric antigen receptor is expressed through transfecting the nucleic acid sequence encoding it into T cells.

Preferably, the transfection is performed by any one of viral vector, eukaryotic expression plasmid or mRNA sequence, or combination of at least two thereof.

Preferably, the viral vector is any one of lentiviral vector, adenovirus vector or retrovirus vector, or a combination of at least two thereof.

In a second aspect, the present invention provides a composition comprising the chimeric antigen receptor described in the first aspect.

In a third aspect, the present invention provides the use of the chimeric antigen receptor as described in the first aspect and/or the composition as described in the second aspect in preparation of chimeric antigen receptor T cells and in drugs for treating tumors.

Compared with the prior art, the present invention has the following beneficial effects:

(1) Through a manner of recognizing pl-CS by VAR2CSA, the chimeric antigen receptor of the present invention allows CAR-T cells to specifically recognize tumor cells, target the tumor microenvironment more easily, enter into the tumor tissues, recognize tumor cells more specifically and have better targetability to tumor microenvironment;

(2) T cells expressing the chimeric antigen receptor of the present invention can kill tumor cells to the maximum extent, having little damage to normal tissues, and can break through the tumor immunosuppressive microenvironment, allowing them to have a better therapeutic effect on solid tumors and be able to treat almost all types of tumors.

### DESCRIPTION OF THE DRAWINGS

FIG. 1 is a schematic view of a CAR lentiviral expression vector targeting pl-CS;

FIG. 2(A) shows the green fluorescence (GFP) expression in T cells transfected with Lentivirus for 96 hours as observed under fluorescence microscope, and FIG. 2(B) shows the GFP positive rate of T cells transfected with Lentivirus for 96 hours as demonstrated by flow cytometry;

FIG. 3(A) shows a comparison of IL-2 concentrations in the supernatants of the experimental group, the negative control group, the positive control group and the wild type T cell group, and FIG. 3(B) shows the comparison of IFN-γ concentrations in the supernatants of the experimental group, the negative control group, the positive control group and the wild type T cell group;

FIG. 4 shows a comparison of the cytotoxicities of the experimental group, the negative control group, and the wild type T cell group against B cell lymphoma cell line Raji under different ratios of effector cells to target cells, wherein the histograms of the experimental group, the negative control group and the wild group are shown from left to right with ratios of effector cells to target cells of 8:1, 4:1, and 1:1, respectively.

### DETAILED DESCRIPTION

In order to further illustrate the technical measures adopted by the present invention and the effects thereof, the technical solutions of the present invention are further described below with reference to the accompanying drawings and specific embodiments, and however, the present invention is not limited to the scope of the embodiments.

### EXAMPLE 1

### Determination of the Gene Sequence of CAR Targeting pl-CS

The sequences of the VAR2CSA gene of Plasmodium falciparum, the signal peptide gene of human CD8α, the hinge region gene of human CD8α, the transmembrane region and intracellular region genes of human CD28, the intracellular region gene of human 4-1BB, and the intracellular region gene of human CD3ζ were retrieved from the GenBank database and literatures. Codon optimization was performed on these genes to ensure that they were more suitable for expressing in human cells without changing the encoded amino acid sequences.

The sequence information of each gene can be seen from SEQUENCE LISTING (SEQ ID NO.1-22 in the Sequence Listing).

### EXAMPLE 2

### Construction of Lentivirus Expression Vector pLHlentiCAR004

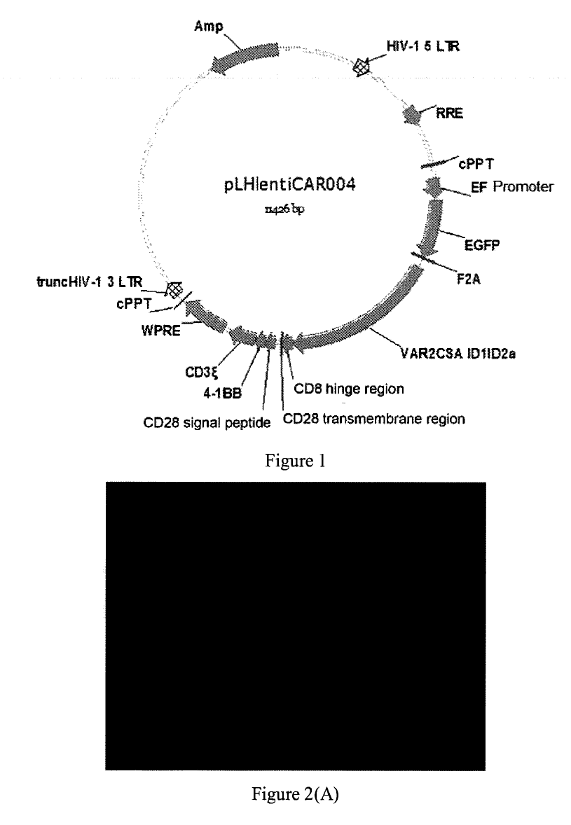

The above gene sequences were sequentially linked in the order of signal peptide gene of human CD8α, VAR2CSA, hinge region gene of human CD8α, transmembrane region and intracellular region genes of human CD28, intracellular region gene of human 4-1BB and intracellular region gene of human CD3ζ to compose the chimeric antigen receptor. The chimeric antigen receptor had a nucleic acid sequence as shown in SEQ ID NO.2. Appropriate restriction enzyme sites were introduced to perform artificial synthesis of the whole gene, which was then cloned into a lentiviral expression vector to obtain pLHlentiCAR004 plasmid. The schematic view of the plasmid was shown in FIG. 1.

The recombinant plasmid was sent to Shanghai Yingjun Biotechnology Co., Ltd. for sequencing. The sequencing result was aligned with the sequence intended to be synthesized, demonstrating that the artificially synthesized sequence was completely correct.

### EXAMPLE 3

### Large-Scale Extraction of Lentiviral Expression Vectors and Packaging Vectors

The stbl3 E. coli harboring pLHlentiCAR004 plasmid and packaging plasmids psPAX2 and pMD2.G was cultured on a large scale in LB medium, and plasmids were extracted on a large scale by alkaline lysis for use in transfection. The specific process was as follows:

1) The stbl3 E. coli strains harboring pLHlentiCAR004, psPAX2, or pMD2.G were inoculated into culture flasks containing 500 mL LB/antibiotic culture medium, respectively, and cultured at 37° C. on a shaker for 12-16 hours;

2) Bacteria were collected by centrifugation at 3000*g for 10 min. The culture medium was discarded and the culture flask was inverted onto absorbent papers and snapped slightly to allow the residual liquid to be absorbed completely. MaxPure Plasmid Maxi Kit from Guangzhou MK Biotech Co., Ltd. was used to extract plasmids on a large scale from the collected bacteria. The specific steps for plasmid extraction were as follows:

(1) 20 mL of Buffer P1/RNase A was added to the bacteria, and the flask was vortexed to re-suspend the bacteria (to ensure that the bacteria were completely re-suspended and the re-suspended solution had no cell aggregate); 20 mL of Buffer P2 was added to the resuspension solution, and inverted for 10-15 times to mix, then resting for 1-2 min (be sure to mix gently, note that the operation time cannot exceed 4 minutes);

(2) 20 mL of Buffer E3 was added to the lysate, and inverted immediately for 15-20 times to mix. The mixing process should be gentle and complete. The standard of complete neutralization was: the solution was no longer viscous and the precipitate was dispersed as white. The resultant was centrifuged at 3000*g for 10 min, then the supernatant was transferred into a new centrifuge tube, 1/3 volume of Buffer E4 was added to the supernatant, vortexed and mixed;

(3) A MaxPure Micro Column was set into a 50 mL centrifuge tube, 15 mL of the mixture was transferred to the column, and centrifuged at 3000*g for 10 min. The effluent was discarded and the column was set back into the collection tube. Next, the remaining solution was transferred into the column and centrifuged at 3000*g for 10 minutes. The effluent was discarded and the column was set back into the collection tube. 20 mL of Buffer E5 was added to the column, and centrifuged at 3000*g for 10 minutes. The effluent was discarded and the column was set back into the collection tube;

(4) 20 mL of Buffer PW2 (diluted with absolute ethanol) was added to the column, and centrifuged at 3000*g for 10 minutes. The effluent was discarded and the column was set back into the collection tube. 20 mL of Buffer PW2 (diluted with absolute ethanol) was added to the column, and centrifuged at 3000*g for 10 minutes. The effluent was discarded and the column was set back into the collection tube, and was centrifuged at 4000*g for 10 minutes.

(5) The column was set into a sterile 50 mL centrifuge tube, and 30-200 mL of sterile water was added to the center of the column membrane, resting for 2 minutes. By centrifugation at 10000*g for 5 min, the separated solution was collected;

(6) Further extraction was performed to remove endotoxin, and the plasmid concentration was adjusted with sterile water to be in the range of 0.1 g-0.6 mg/mL;

(7) 0.1 volume of Buffer ER1 and 0.1 volume of Buffer ER2 were added to the plasmid solution, and were inverted to mix. Then the mixture was placed on ice for 20 min, during which it was inverted and mixed for several times. Next, it was placed in 42° C. water bath for 5 min, and then centrifuged at 10000*g at room temperature for 3 min;

(8) The sample was taken out gently and a layer of red solution was found to form at the bottom. The supernatant was carefully transferred into a centrifuge tube, 0.7 volume of isopropyl alcohol was added to the supernatant, and inverted for several times to mix. The mixture was centrifuged at a speed of 10000*g at room temperature for 10 min, and the supernatant was discarded. 1 volume of 70% ethanol was added to the precipitate, vortexed and mixed for 15-30 seconds. The resulting mixture was centrifuged at 10000*g at room temperature for 3 minutes, and the supernatant was carefully discarded. Centrifuged at 10000*g at room temperature for additional 1 minute to collect droplets from the tube wall. Do not aspirate the precipitate. Dry in the air for 10 min;

(9) An appropriate amount of sterile water was added to the plasmid, vortexed for 10-20 seconds, and placed at room temperature for 30 minutes to allow the plasmid to fully dissolve, during which the mixture was inverted and mixed for several times;

The plasmid concentration was measured by a Nanodrop UV spectrophotometer and the plasmid was then stored at -20° C.

### EXAMPLE 4

### Packaging, Concentration and Titration of Lentivirus

1) Packaging of Lentivirus

(1) Pre-plating of cells: 24 hours before transfection, 293T cells that were overgrown were pre-plated so that 70% confluence could be reached next day (pre-plating 5 million cells in a 75 mm dish).

(2) Day 2: Transfection

(a) The pre-plated cells were observed for growth. When the cells grew to about 90% confluence, the medium was discarded and freshly pre-warmed complete culture medium was added (15 mL in a 75 mm dish);

(b) PEI, plasmid and Opti-MEM I Reduced Serum Medium, etc. required for transfection were placed at room temperature for balancing prior to transfection;

(c) Preparation of mixture of plasmids: A total 10 dishes of 293T cells were packaged this time. 500 μL of mixture should be prepared for each dish, and thus a total volume of 5 mL of mixture should be prepared. Firstly, three types of plasmid were added respectively according to the usage amount of the individual plasmid, with a total of 420 μg (pLHlentiCAR004: psPAX2: pMD2.G=3:2:1), and Opti-MEM I Reduced Serum Medium was added to adjust the volume to 2.5 mL, mixed by blowing, and placed at room temperature for 10 min. Then 1.25 mL of PEI and 1.25 mL of Opti-MEM I Reduced of Serum Medium were mixed and placed at room temperature for 10 min. Finally, the above two mixture solutions were mixed and placed at room temperature for 10 min to obtain a mixture used for transfection;

(d) The mixture was added dropwise to the culture dish at 500 μL per dish;

(3) After transfection for 6 h, the medium in each dish was changed to 17 mL of UltraCLUTURE serum-free medium;

(4) Viruses were harvested between Day 3 and Day 5: after transfection for 48 h-72 h, the culture supernatants were collected respectively. The collected viral supernatants were temporarily stored at 4° C.

2) Concentration of Lentivirus

The collected culture supernatants were filtered through a 0.45 um membrane, followed by centrifuging with a SW-32Ti rotor at 25000 rpm (corresponding to a centrifugal force of 106750 g) at 4° C. for 2 h. After removing the supernatants, the pellets were re-suspended with 200 μl of PBS, and stored at −80° C.

3) Titration of Lentivirus

(1) 293T cells were pre-plated into 6-well plates, 3×10⁵ cells/well, and incubated at 37° C. in a 5% CO₂ incubator overnight (for approximately 18-20 h);

(2) Preparation of a stock solution of polybrene: sterilized pure water, 8 mg/mL, filtered with a 0.22 μm filter tip, sub-packaged and stored at −20° C. for later use.

(3) Preparation of polybrene-containing DMEM medium: 10% FBS, 1% double antibody, 8 μg/mL polybrene (one thousandth of the volume of the medium);

(4) Thawing of viruses: The virus frozen stock was taken out from −80° C., and thawed at room temperature. The thawed viruses were placed on ice and taken into the cytology lab for use.

(5) Dilution of virus: The virus stock solution was diluted 10-fold with 5% FBS DMEM complete culture medium containing polybrene to obtain a diluted virus solution of 10⁻¹-10⁴. Note that the virus solution should be mixed uniformly before taking the solution every time for a further dilution.

(6) The original medium was discarded. 1 mL of virus-free medium was added to the first well as a negative control, and 1 mL of the corresponding virus dilution was added to each of the remaining wells and incubated at 37° C. for 18-20 h;

(7) The virus-containing medium was removed the next morning and replaced with 2 mL of 5% FBS DMEM medium without polybrene. The cells were observed under a microscope for the expression of GFP;

(8) The medium was aspirated, and then the cells were digested with trypsin. The cells were blown into single cells, and an appropriate amount of complete culture medium was added to stop the trypsin reaction. The cells were collected by centrifugation at 300 g for 3 min;

(9) The supernatant was decanted, the pellet was re-suspended with an appropriate amount of pre-cooled PBS, followed by centrifugation at 300 g for 3 min, and the cells was collected. The above procedure was repeated for one more time;

(10) Finally, the pellet was re-suspended with 1 mL of pre-cooled PBS and the suspension was placed on ice for flow cytometry analysis.

(11) The wells with a GFP positive rate of 1%-20% were selected, and the virus titer of each well was calculated, and finally averaged;

titer={(F×Cn)/V}×DF, with a unit of TU/mL; calculation formula:

wherein: F: the rate of GFP positive cells; Cn: number of cells plated per well; V: volume of virus dilution added; DF: virus dilution factor

The virus titer was calculated to be in the range of: 1.6×10⁸-1×10¹⁰.

### EXAMPLE 5

### Isolation, Culture and Infection of T Cells

1) Isolation of PBMC cells

(1) 50 mL of blood was collected and centrifuged at 800 g for 10 min (acceleration was set at 3, speed reduction was set at 4);

(2) Leukocyte layer was taken and diluted to 8 mL with 2% FBS;

(3) 4 mL LymphoPrep was added into a 15 mL centrifuge tube, then the diluted blood was carefully added onto the stratification solution along the tube wall to keep a clear interface between the two solutions;

(4) Centrifuged at 800 g for 20 min (acceleration was set at 6, speed reduction was set at 1);

(5) The mononuclear cells with a color of gray white were gently aspirated by using a Pasteur pipette, added to another centrifuge tube already containing 10 mL RF-10, and mixed uniformly;

(6) Centrifuged at 500 g for 5 min and the supernatant was discarded;

(7) The cells were re-suspended with 10 mL of RF-10, counted by trypan blue, and centrifuged at 350 g for 10 min, and the supernatant was discarded.

2) Magnetic Bead Sorting of CD4+T (CD8+T) Cells

(1) Magnetic beads were vortexed and mixed and 25 μL of beads were taken into a test tube. 1 mL of Buffer 1 was added and mixed. Then the test tube was placed on magnetic rack for 1 min. The supernatant was discarded. 254, of Bffer1 was added to re-suspend the magnetic beads for use;

(2) PBMCs were re-suspended with Buffer 1 to a density of 10⁷ cells/mL;

(3) 25 μL of the washed magnetic beads was added to 1 mL of PBMCs and incubated at 2-8° C. for 20 min, by rotating obliquely on a shaking bed;

(4) The test tube was placed on a magnet for 2 min and the supernatant was collected;

(5) The test tube was removed, 1 mL of Buffer 1 was added, mixed by blowing, placed on the magnetic beads for 2 min and the supernatant was collected, all of which were repeated once;

(6) 100 μL of Buffer 2 was added to re-suspend the cells, 10 μL of DETCHaBEAD was added, and incubated at room temperature for 45 min to allow the cells to release from the magnetic beads;

(7) The test tube was placed on the magnetic rack for 1 min and the supernatant containing cells was transferred to a new test tube. 500 μL of Buffer 2 was added to wash the beads for 2-3 times, and the supernatant was collected;

(8) 4 mL of Buffer 2 was added and centrifuged at 350 g for 5 min, the supernatant was discarded, and the cells were re-suspended with Buffer 2;

(9) CD8+ T cells were collected from the supernatant collected during the sorting of CD4+ T cells according to the instruction of kit.

3) Culture of T Cells

(1) CD4+T (CD8+T) cells sorted by magnetic beads were centrifuged at 350 g for 10 min;

(2) The cells were re-suspended with RF-10 and counted;

(3) CD4+T and CD8+T cells were added to culture plates at a ratio of 1:1 for culture, with a cell density of 5×10⁵ cells/mL;

(4) CD3/CD28 antibody magnetic beads were added to the T-cell specialized medium at a ratio of magnetic beads to cells of 1:1;

(5) rhlL-2 was added at a final concentration of 10 ng/mL;

(6) Cells were counted 2-3 times per week and were recorded for the proliferation.

4) Lentivirus Transfection of T Cells.

(1) CD4+T (CD8+T) cells sorted by magnetic beads were centrifuged at 350 g for 10 min. The cell pellets were re-suspended in complete culture medium before cell counting.

(2) CD4+T and CD8+T cells were added to 96-well plates at a ratio of 1:1 for culture, with a cell density of 5×10⁵ cells/mL, and with cell number per well of 1×10⁵;

(3) The magnetic beads were washed before adding to culture dish at a bead-to-cell ratio of 1:1;

(4) rhlL-2 was added at a final concentration of 10 μg/L;

(5) After 24 hours of stimulation, the cells were infected, and polybrene was added at a final concentration of 6 μg/mL, and mixed;

(6) The medium was changed after 16-24 hours;

(7) The cells were imaged with a fluorescence microscope three days later.

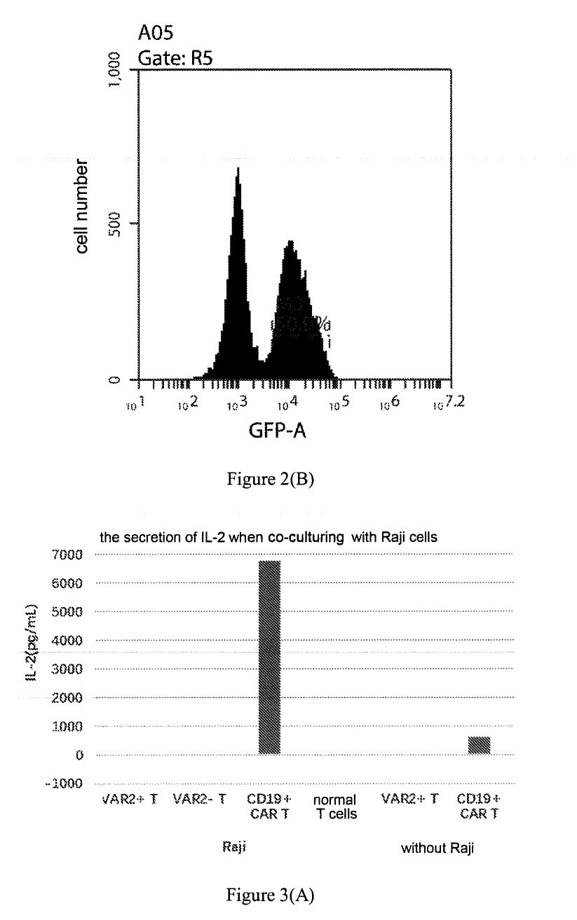

The result of fluorescence microscopy was shown in FIG. 2(A) and the infection efficiency detected by flow-cytometry was shown in FIG. 2(B). It can be seen that the infection efficiency detected by flow-cytometry was about 50%, and CAR-T cells can be efficiently produced.

### EXAMPLE 6

### In Vitro Release and Detection of Cytokines of CAR-T Cells

(1) In a 48-well plate, CAR-T cells, 1.5×10⁵ per well, were co-cultured with Huh7 cells, 1.5×10⁵ cells per well, for 24 hours, and 500 μL of RPMI-1640 culture medium (without serum and rhlL-2) was added;

(2) Collection and storage of samples: the supernatant was collected by centrifugation, and used for experiment directly or sub-packaged and stored at −20° C. (100 μL per tube);

(3) Preparation of reagents: all the reagents were taken out from storage and allow them to warm to room temperature. Wash Buffer concentrate was diluted to 500 mL with deionized water. A and B solutions were mixed in equal volumes to give a color-substrate solution, which should be used within 15 minutes, and at an amount of 200 μL per well. For cytokine standards, 500 μL stock solution was taken and diluted into 6 concentrations in gradient;

(4) Samples and the gradiently diluted standards were respectively added to ELISA plates which had been coated with antibody at an amount of 100 μL per well, removing air bubbles, and incubated at room temperature for 2 h (three replicates were set for each group of samples);

(5) Fluid in the wells was removed, 300 μL of WB was added into each well for washing, the washing was repeated for three times, and the WB was completely aspirated at the last washing;

(6) 200 μL of corresponding detection antibodies were added and incubated for 2 h at room temperature;

(7) Fluid in the wells was removed, 300 μL of WB was added into each well for washing, the washing was repeated for three times, and the WB was completely aspirated at the last washing;

(8) 200 μL of substrate solution was added and incubated at room temperature for 20 min (dark light);

(9) 50 μL of stop solution was added. The color of the solution was changed from blue to yellow. If the color is green or doesn't change, tap slightly to mix the solution;

(10) Absorbance was measured at 450 nm within 30 min;

(11) Concentrations of various cytokines were calculated according to standard curves,

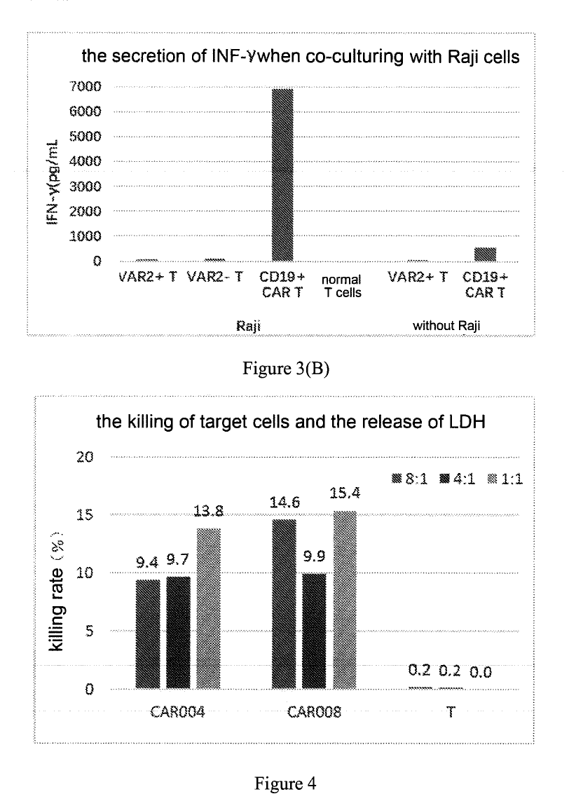

The results were shown in FIGS. 3(A) and 3(B), from which it can be seen that CAR-T cells do not release cytokines when co-culturing with tumor cells, suggesting that the CAR-T cells have no adverse effect of strong cytokine storms, and thus will not cause side effects such as inflammatory reactions.

### EXAMPLE 7

### In Vitro Killing Tumor Cells by CAR-T Cells

1) Determination of the Optimal Cell-Plating Concentrations

(1) Various tumor cells were collected, washed with assay medium, and adjusted to a concentration of 1×10⁵ cells/mL;

(2) 100 μL of assay medium was added to 96-well plate and bespread;

(3) 100 μL of cell suspension was added to the 96-well plate and serial dilution was performed. 100 μL of cell suspension was aspirated from the final concentration gradient. Six duplicate wells were set with three wells being High control group and three wells being Low control group, and three wells containing assay medium without cells were set as Background control (range, 500-50000);

(4) Incubated in an incubator for 18 hours;

(5) 5 μL of cell lysate was added to each well of the HC group and incubated for 15 min. Shook to accelerate cell lysis.

(6) 100 μL of freshly prepared reaction solution was added to each well and incubated at 15° C.-25° C. for 30 minutes (note that the 96-well plate should be protected from light);

(7) 50 μL of stop solution was added to each well and shook for 10 s;

(8) Absorbance was measured at 490 nm;

(9) The data was analyzed to determine the optimal cell concentration (maximum difference between HC and LC).

2) Detection of Cell-Mediated Toxicity

(1) CAR-T cells were collected and washed with assay medium;

(2) The number of CAR-T cells to be plated in a 96-well plate was determined according to different ratios of effector cells to target cells and the optimal concentrations as the results in 1);

(3) The corresponding cell suspension to be plated in the 96-well plate was adjusted to 50 μL volume per well.

(4) Various tumor cells were collected and the cell concentrations were adjusted to 2 times of the optimal concentrations. 50 μL of cell suspension was added into T cells;

(5) Different control groups were set up;

(6) Incubated in an incubator at 37° C. for 18 h;

(7) 5 μL of cell lysate was added to each well and incubated for 15 min. Shook to accelerate cell lysis.

(8) 100 μL of freshly prepared reaction solution was added to each well and incubated at 15° C.-25° C. for 30 minutes (note that the 96-well plate should be protected from light);

(9) 50 μL of stop solution was added to each well and shook for 10 s;

(10) Absorbance was measured at 490 nm;

(11) Each sample was calculated for the percentage of cytotoxicity.

The results were shown in FIG. 4. Under different ratios of effector cells to target cells, CAR-T cells have toxicity on tumor cells, and can efficiently kill tumor cells and release LDH. However, wild type T cells, as a control group, cannot kill tumor cells.

In summary, T cells expressing the chimeric antigen receptor of the present invention can kill tumor cells to the maximum extent, without releasing of cytokines, having little damage to normal tissues, and can break through the tumor immunosuppressive microenvironment, allowing them to have a better therapeutic effect on solid tumors and be able to treat almost all types of tumors.

The applicant states that detailed methods of the present invention are demonstrate in the present invention through the above embodiments, however, the present invention is not limited to the above detailed methods, and does not mean that the present invention must rely on the above detailed methods to implement. It should be apparent to those skilled in the art that, for any improvement of the present invention, the equivalent replacement of the raw materials of the present invention, the addition of auxiliary components, and the selection of specific modes, etc., will all fall within the protection scope and the disclosure scope of the present invention.

1. A chimeric antigen receptor of anti-placental-like chondroitin sulfate, mainly comprising an antigen recognition region of anti-pl-CS, a hinge region, a transmembrane region, and an intracellular region in tandem arrangement;
wherein, the antigen recognition region of anti-pl-CS is any one of Plasmodium falciparum protein VAR2CSA, partial peptide fragment of the Plasmodium falciparum protein VAR2CSA, or antibody against pl-CS; and
the partial peptide fragment of the Plasmodium falciparum protein VAR2CSA is a peptide fragment having a number of amino acids greater than 500 from the Plasmodium falciparum protein VAR2CSA.

2. The chimeric antigen receptor according to claim 1, wherein the Plasmodium falciparum protein VAR2CSA is a VAR2CSA protein of Plasmodium falciparum strain pf 3D7 and/or a VAR2CSA protein of Plasmodium falciparum strain pf FCR3;

3. The chimeric antigen receptor according to claim 1, wherein the hinge region is the hinge region of human CD8α.

4. The chimeric antigen receptor according to claim 1, wherein the transmembrane region is the transmembrane region of human CD28.

5. The chimeric antigen receptor according to claim 1, wherein the intracellular region is any one of the intracellular region of human CD3ζ, the intracellular region of human CD28, or the intracellular region of human 4-1BB, or a combination of at least two thereof.

6. The chimeric antigen receptor according to claim 1 wherein the chimeric antigen receptor further contains a human CD8α signal peptide at its amino terminus.

7. The chimeric antigen receptor according to claim 1, wherein the chimeric antigen receptor is composed of human CD8α signal peptide, VAR2CSA, hinge region of human CD8α, transmembrane region and intracellular region of human CD28, intracellular region of human 4-1BB and intracellular region of human CD3ζ in tandem arrangement.

8. The chimeric antigen receptor according to claim 1 wherein the chimeric antigen receptor is expressed through transfecting the nucleic acid sequence encoding it into T cells.

9. A composition comprising the chimeric antigen receptor according to claim 1.

10. A method for preparing chimeric antigen receptor T cells, comprising expressing the chimeric antigen receptor according to claim 1 in T cells.

11. A method for treating tumors, comprising administrating the chimeric antigen receptor according to claim 1.

12. The chimeric antigen receptor according to claim 2, wherein the VAR2CSA protein of Plasmodium falciparum strain pf 3D7 has an amino acid sequence of SEQ ID NO.3, and a nucleic acid sequence of SEQ ID NO.4.

13. The chimeric antigen receptor according to claim 2, wherein the VAR2CSA protein of Plasmodium falciparum strain pf FCR3 has an amino acid sequence of SEQ ID NO.5, and a nucleic acid sequence of SEQ ID NO.6.

14. The chimeric antigen receptor according to claim 1, wherein the partial peptide fragment of the Plasmodium falciparum protein VAR2CSA is ID1-ID2a, which has an amino acid sequence of SEQ ID NO.7, and a nucleic acid sequence of SEQ ID NO.8.

15. The chimeric antigen receptor according to claim 3, wherein the hinge region of human CD8a has an amino acid sequence of SEQ ID NO.9, and a nucleic acid sequence of SEQ ID NO.10.

16. The chimeric antigen receptor according to claim 4, wherein the transmembrane region of human CD28 has an amino acid sequence of SEQ ID NO.11, and a nucleic acid sequence of SEQ ID NO.12.

17. The chimeric antigen receptor according to claim 5, wherein the intracellular region is the intracellular region of CD3ζ, the tandem arrangement of the intracellular region of human CD28 and the intracellular region of human CD3ζ, the tandem arrangement of the intracellular region of human 4-1BB and the intracellular region of human CD3ζ, or the tandem arrangement of the intracellular region of human CD28, the intracellular region of human 4-1BB and the intracellular region of human CD3ζ.

18. The chimeric antigen receptor according to claim 17, wherein the intracellular region of human CD3ζ has an amino acid sequence of SEQ ID NO.13, and a nucleic acid sequence of SEQ ID NO.14.

19. The chimeric antigen receptor according to claim 17, wherein the tandem arrangement of the intracellular region of human CD28 and the intracellular region of human CD3ζ has an amino acid sequence of SEQ ID NO.15, and a nucleic acid sequence of SEQ ID NO.16.

20. The chimeric antigen receptor according to claim 17, wherein the tandem arrangement of the intracellular region of human 4-1BB and the intracellular region of human CD3 has an amino acid sequence of SEQ ID NO.17, and a nucleic acid sequence of SEQ ID NO.18.

21. The chimeric antigen receptor according to claim 17, wherein the tandem arrangement of the intracellular region of human CD28, the intracellular region of human 4-1BB and the intracellular region of human CD3ζ has an amino acid sequence of SEQ ID NO.19, and a nucleic acid sequence of SEQ ID NO.20.

22. The chimeric antigen receptor according to claim 6, wherein the human CD8α signal peptide has an amino acid sequence of SEQ ID NO.21 and a nucleic acid sequence of SEQ ID NO.22.

23. The chimeric antigen receptor according to claim 7, wherein the chimeric antigen receptor has an amino acid sequence of SEQ ID NO.1 and a nucleic acid sequence of SEQ ID NO.2.

24. The chimeric antigen receptor according to claim 8, wherein the transfection is performed by any one of viral vector, eukaryotic expression plasmid, or mRNA sequence, or combination of at least two thereof.

25. The chimeric antigen receptor according to claim 24, wherein the viral vector is any one of lentiviral vector, adenovirus vector, or retrovirus vector, or a combination of at least two thereof.
